(19) United States
(12) Patent Application Publication
(10) Pub. No.: US 2019/0029652 A1
(43) Pub. Date: Jan. 31, 2019

(54) Urine Sample Collection Device

(71) Applicant: Jonathan Flake, Provo, UT (US)

(72) Inventor: Jonathan Flake, Provo, UT (US)

(21) Appl. No.: 15/662,157

(22) Filed: Jul. 27, 2017

(51) Int. Cl. A61B 10/00

(52) U.S. Cl. CPC A61B 10/007

(57) ABSTRACT

A urine sample collection device comprises an elongated arm having a proximal end defining a handle and an opposite distal end. A funnel extends from the arm at the distal end and has a solid conical wall circumscribing a larger inlet opening, and has a smaller outlet opening. An open loop extends from the arm at the distal end, under and spaced-apart from the outlet opening of the funnel, and having an opening therein. The open loop removably receives a urine sample cup through the opening, and holds the urine sample cup underneath and spaced-apart from the outlet opening. The funnel and the open loop being sized and shaped to form a sanitary gap to resist contact between the urine sample cup and genital tissue.

### BACKGROUND

### Field of the Invention

The present invention relates generally to collecting urine samples.

### Related Art

Urine samples are typically collected as part of routine medical examinations, particularly for pregnant women. It can be difficult to effectively obtain the sample. Various elongated handles have been proposed to help position a sample cup to receive the sample, some of which mount to a toilet seat. For example, see U.S. Pat. Nos. 8,091,848; 8,597,207; 6,299,606; 5,920,916; 5,492,220; and D773,659; and US Patent Application Publications 2015/0223783; 2002/0169395; and 2015/0297194. In order to properly analyze the urine sample, the sample must be sterile and/or uncontaminated. One source of contamination is contact between genital tissue and the sample cup.

### SUMMARY OF THE INVENTION

It has been recognized that it would be advantageous to develop a urine sample collection device that facilitates placement of a sample cup to receive the urine sample, and to do so in a manner to facilitate a sterile, sanitary and/or uncontaminated sample, and resist contact between the genital tissue and the sample cup.

The invention provides a urine sample collection device with an elongated arm having a proximal end defining a handle and an opposite distal end. A funnel extends from the arm at the distal end and has a solid conical wall circumscribing a larger inlet opening, and has a smaller outlet opening. An open loop extends from the arm at the distal end, under and spaced-apart from the outlet opening of the funnel, and having an opening therein. The open loop removably receives a urine sample cup through the opening, and holds the urine sample cup underneath and spaced-apart from the outlet opening. The funnel and the open loop being sized and shaped to form a sanitary gap to resist contact between the urine sample cup and genital tissue.

In addition, the invention provides a method for collecting a urine sample, comprising: holding a urine sample collecting device; inserting a urine sample cup through the opening of the open loop and into the open loop with the urine sample cup underneath and spaced-apart from the outlet opening of the funnel; positioning the inlet opening of the funnel adjacent to and facing upwardly and forwardly to the genitalia, and with the funnel and the urine sample cup pendent from the arm, and with the open loop oriented substantially horizontally corresponding to an upright orientation of the urine sample cup; collecting urine from the genitalia through the funnel and into the urine sample cup with the funnel and the open loop and/or the sample cup forming the sanitary gap to resist contact between the urine sample cup and the genital tissue.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of a urine sample collection device in accordance with an embodiment of the present invention;

FIG. 2 is a partial side view of the urine sample collection device of FIG. 1, shown with a urine sample cup;

FIG. 3 is a partial from view of the urine sample collection device of FIG. 1 shown with the urine sample cup;

FIG. 4 is a side view of the urine sample collection device of FIG. 1;

FIG. 5 is a front view of the urine sample collection device of FIG. 1;

FIG. 6 is a top view of the urine sample collection device of FIG. 1;

FIG. 7 is a rear view of the urine sample collection device of FIG. 1;

FIG. 8 is a bottom view of the urine sample collection device of FIG. 1;

FIG. 9 is a side cross-sectional view of the urine sample collection device of FIG. 1, taken along line 9 of FIG. 5;

FIG. 10 is a side view of the urine sample collection device of FIG. 1.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

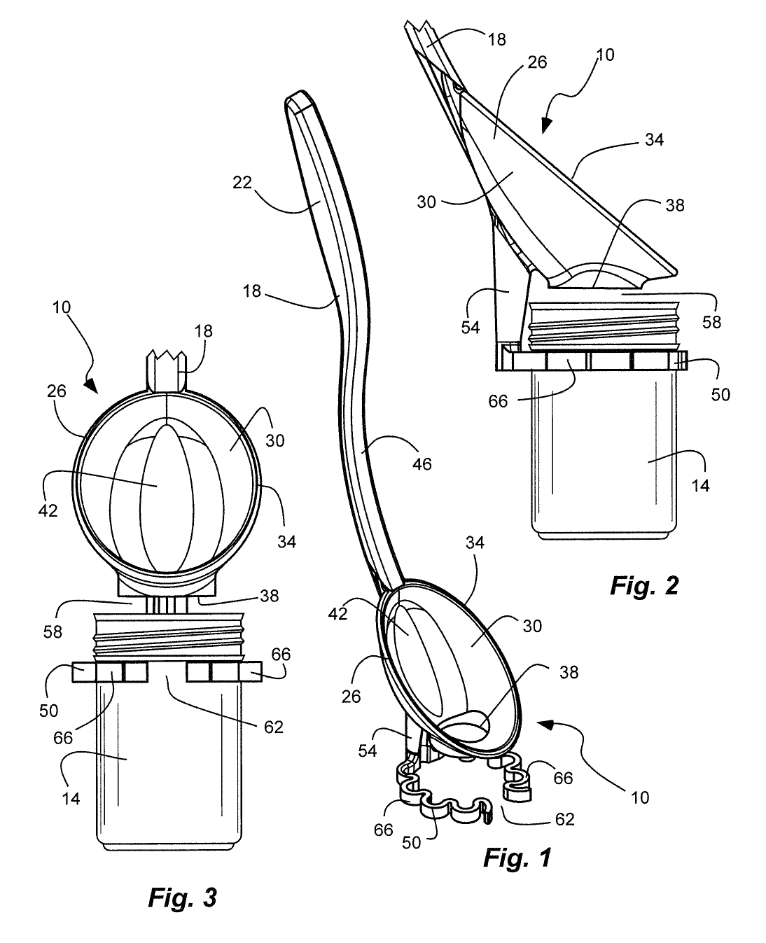

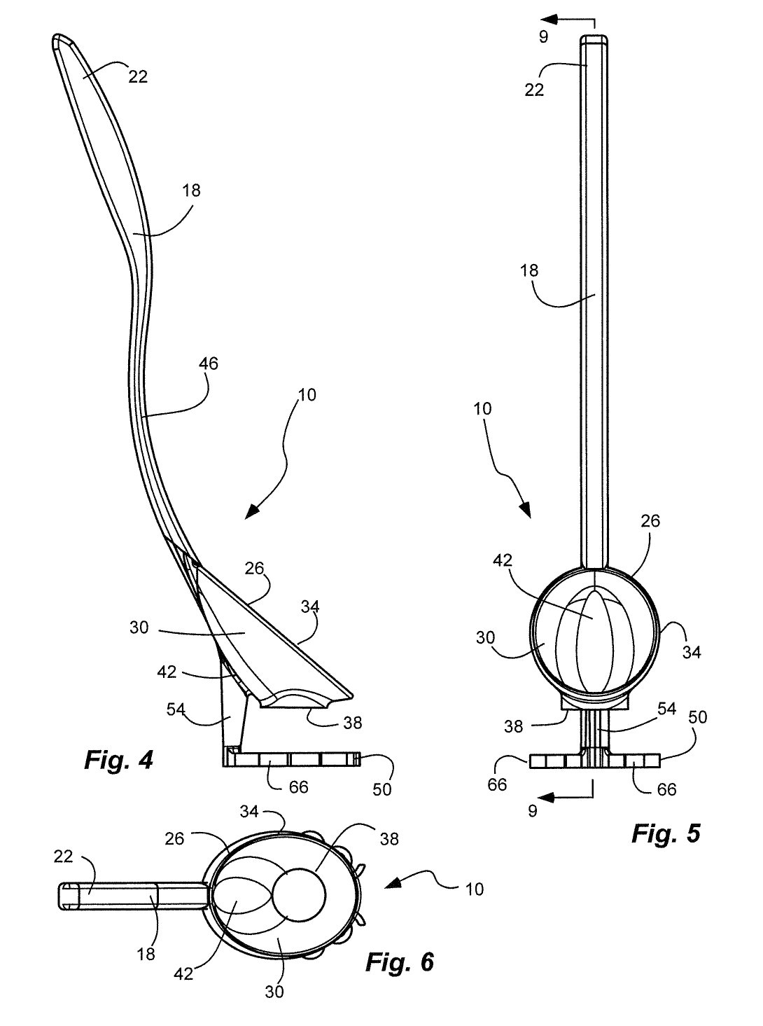

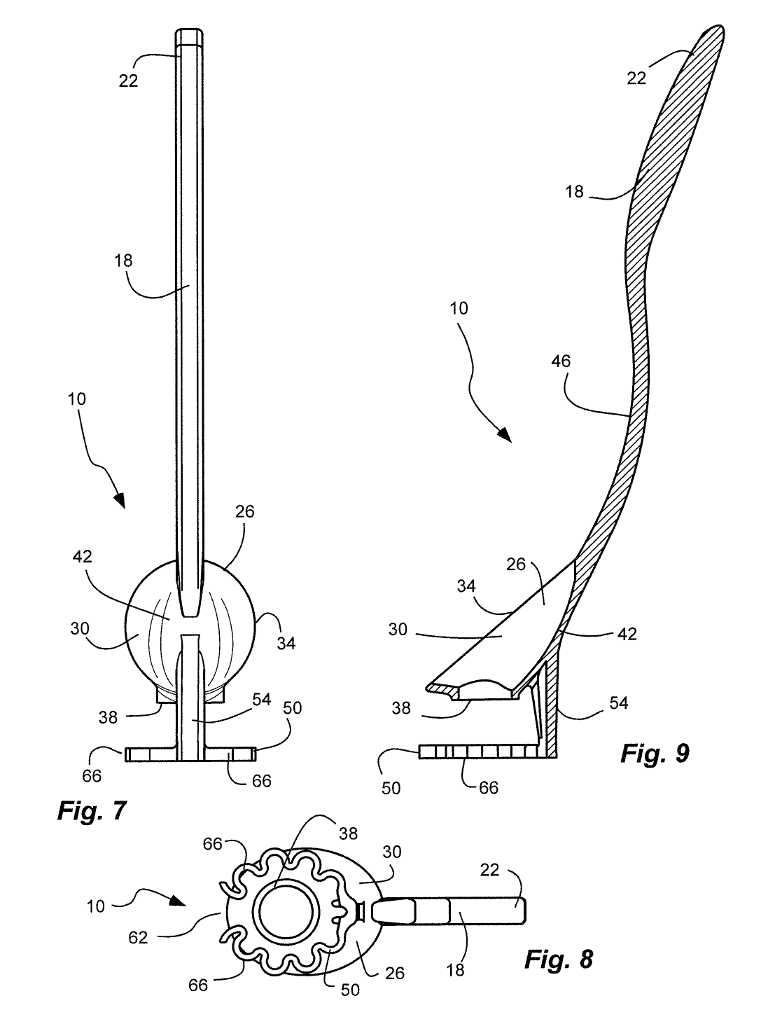

As illustrated in FIGS. 1-10, a urine sample collection device, indicated generally at 10, in an example implementation in accordance with the invention is shown for facilitating collection of urine from a user or patient into a urine sample cup 14. In one aspect, the urine sample cup 14 can be a standard size and shape, such as 4 oz. (120 cc) cup with a locking threaded opening. The urine sample collection device 10 can be particularly well suited for use by female users or patients, and pregnant users or patients. The urine sample collection device 10 can facilitate a sanitary condition of the sample in the urine cup 14 by providing a sanitary gap between the urine sample cup 14 and genital tissue of the user. Thus, genital tissue is less likely to contact the urine sample cup 14 and contaminate the sample.

The collection device 10 has an elongated arm 18 with a proximal end that can be held by the user or patient, and an opposite distal end. The proximal end can have or can define a handle 22. A funnel 26 extends from, is disposed on, and is carried by the distal end of the arm 18. The funnel 26 has a solid conical wall 30 circumscribing a larger inlet opening 34. In addition, the funnel 26 also has a smaller outlet opening 38. The outlet opening 38 of the funnel 26 can be smaller than the opening of the sample cup 14. The funnel 26 and the arm 18 can share a wall (extending between the arm 18 and an open loop described below) and extending through the funnel. In one aspect, the arm 18 extends from the handle 22 and opens into the funnel 26.

In one aspect, the funnel 26 is irregular with the outlet opening 38 being off-axis, and non-parallel, with respect to the inlet opening 34. The funnel 26 can be an oblong frustum, or oblong frusto-conical shell, with a top or larger end of the frustum having the inlet opening 34, and a bottom or smaller end of the frustum being substantially closed, or having a closed bottom 42. The outlet opening 38 can be formed in a side of the frustum or the conical wall 30. Thus, the funnel 26 can form an angularly inclined bowl with the inlet opening 34 being angularly inclined with respect to horizontal (or with respect to the open loop when the open loop is oriented horizontally to hold the cup in an upright orientation). Thus, the inlet opening 34 faces upwardly and forwardly with respect to horizontal (or with respect to the open loop when the open loop is oriented horizontally corresponding to an upright orientation of a urine cup), and can face the genitalia of the user or patient.

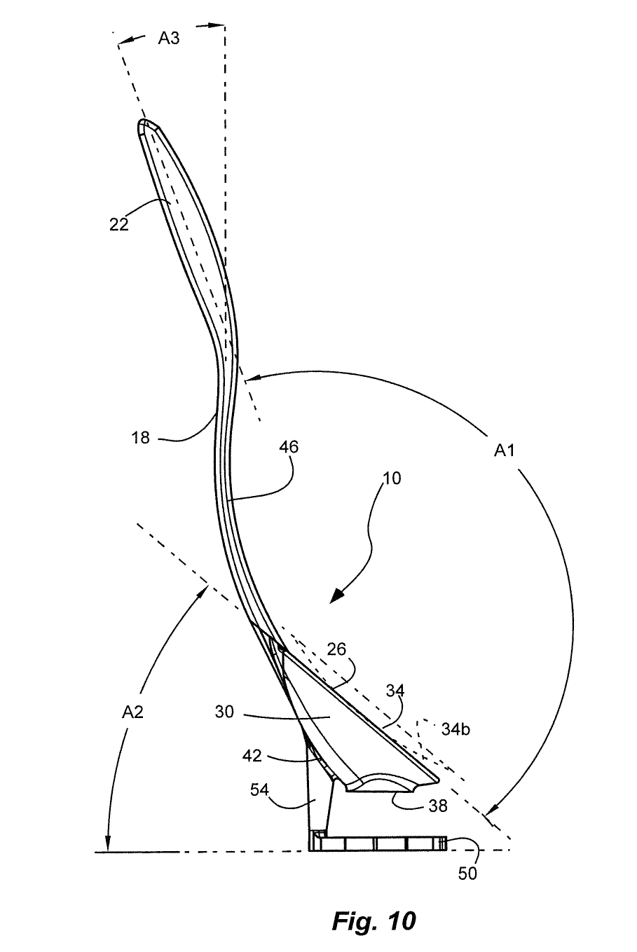

The collection device 10 can be configured to facilitate the position and orientation of the funnel 26 for ease of use. The inlet opening 34 and the arm 18, or handle 22 or proximal end thereof, can be oriented at an obtuse angle A1, between approximately 130 to 180 degrees in one aspect, between approximately 140 to 170 degrees in another aspect, between approximately 150 to 160 degrees in another aspect, or at approximately 155 degrees, as shown in FIG. 10. The inlet opening 34 can be oriented at an acute angle A2 with respect to horizontal (or the open loop) between approximately 30 to 50 degrees in one aspect, or at an acute angle of approximately 40 degrees in another aspect, as shown in FIG. 10. The outlet opening 38 (and the open loop) can be oriented substantially horizontally during use to maintain the sample cup 14 in an upright orientation and facilitate flow of urine from the funnel 26 and into the sample cup 14. The inlet opening 34 and the outlet opening 38 can be oriented at acute angle, also represented by A2, with respect to one another, and as described above. While the funnel 26 can be configured to face upward and forwardly towards the genitalia during use, the arm 18 can be configured to facilitate holding during use. In one aspect, the arm 18, or handle 22 or proximal end thereof, can be oriented substantially vertically or inclined, and can form an acute angle A3 up to 20 degrees from vertical, when in use (and when the open loop is oriented horizontally), as shown in FIG. 10. Thus, the handle 22 can be held near the user's or patient's chest or torso during use, rather than forwardly near the thigh or knee, during use. In another aspect, the handle 22 and the arm 18 can be held at an incline, such as approximately 45 degrees. In addition, the funnel 26 and the sample cup 14 (and the open loop) can be pendent from the arm 18 and the handle 22 during use. In one aspect, the collection device and the arm 18 can have an intermediate bow 46 formed in the arm 18 between the handle 22 and the funnel 26, and forming a concave arc with respect to the handle and the funnel (and the user or patient), and defining a belly indentation. The belly indentation can facilitate use of the collection device 10 by a pregnant user or patient, with the handle 22 still held near the chest or torso, and the funnel and collection cup pendent therefrom.

As indicated above, the collection device 10 also has an open loop 50 to removably receive and hold the collection cup 18. The open loop 50 extends from, is disposed on, and is carried by the arm 18 at the distal end. In one aspect, a spar 54 can extend from the funnel 26, and the wall 30 or the closed bottom 42 thereof, to the open loop 50, with the open loop 50 pendent from the spar 54 and the funnel 26. The open loop 50 positions the sample cup 14 under and spaced-apart from the outlet opening 38 of the funnel 26, thus forming a sanitary gap 58 between the sample cup 14 and the funnel 26 and the genital tissue of the user or patient. In addition, the open loop 50 can have an opening 62 therein to removably receive the sample cup 14 through the opening. The open loop 50 can then removably hold the sample cup 14 underneath and spaced-apart from the smaller outlet 38 of the funnel 26. The open loop 50 can be configured to removably receive the sample cup 14 through the opening 62. In one aspect, the open loop 50 can comprise a pair of fingers 66. Each finger 66 can form a serpentine arc, extending inwardly and outwardly along the arc from a proximal attached end, attached to the spar 54 or the arm 18, to a distal free end. Each finger 66 can have a constant cross-sectional size and shape along a length of the finger so that both inner and outer sides of the finger are serpentine. A material of the finger and the serpentine shape can allow the finger to bend or flex outwardly to accept the sample cup 14 through the opening 62, and grip the sample cup in the open loop. The distal ends of the fingers 66 can arc outwardly to provide a curved surface leading into the open loop that has a wider outer opening and a narrower inner opening.

The entire collection device 10, including the arm 18, the funnel 26, the open loop 50 (and the spar 54) can be integrally formed at the same time as a single, monolithic body. In one aspect, the collection device 10 can be formed of plastic, and can be formed by injection molding. In another aspect, the collection device 10 can be disposable. In another aspect, the collection device 10 can be provided in a sterile, sanitary and hygienic manner, such as a flexible sterile wrapper. The sterile wrapper can resist contamination of the funnel during shipping and handling, and thus the sample during use.

A method for collecting a urine sample, and for using the collection device 10 described above, comprises: holding the urine sample collecting device 10; inserting a urine sample cup 14 through the opening 62 of the open loop 50 and into the open loop with the urine sample cup underneath and spaced-apart from the outlet opening 38 of the funnel 26; positioning the inlet opening 34 of the funnel 26 adjacent to and facing upwardly and forwardly to the genitalia, and with the funnel 26 and the urine sample cup 14 pendent from the arm 18, and with the open loop 50 oriented substantially horizontally corresponding to an upright orientation of the urine sample cup 14; collecting urine from the genitalia through the funnel 26 and into the urine sample cup 14 with the funnel 26 and the open loop 50 and/or the sample cup 18 forming the sanitary gap 58 to resist contact between the urine sample cup 18 and the genital tissue.

In one aspect, the inlet opening 34 can have a flat or planar perimeter, as shown in the drawings, and defining a plane that forms an acute angle A2 with respect to horizontal or the open loop 50, and the obtuse angle A1 with the arm 18, or handle 22. In another aspect, the inlet opening 34b can be broadly curved, as shown by dashed lines in FIG. 10. The curved inlet opening 34b can still face upwardly and forwardly, and can define a plane, shown by the dashed line in FIG. 10, that is a mean or average plane of the curvature, or that extends from a top to a bottom of the opening, and that still forms an acute angle A2 and the obtuse angle A1.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A urine sample collection device, comprising:
a) an elongated arm having a proximal end defining a handle and an opposite distal end;
b) a funnel extending from the arm at the distal end and having a solid conical wall circumscribing a larger inlet opening, and having a smaller outlet opening;
c) an open loop extending from the arm at the distal end, under and spaced-apart from the outlet opening of the funnel, and having an opening therein, the open loop configured to removably receive a urine cup through the opening and hold the urine cup underneath and spaced-apart from the smaller outlet.

2. The device in accordance with claim 1, wherein the funnel is irregular with the outlet opening being off-axis and non-parallel with respect to the inlet opening.

3. The device in accordance with claim 1, wherein the funnel is an oblong frustum with a top of the frustum having the inlet opening and a bottom of the frustum being substantially closed, and with the outlet opening formed in a side of the frustum.

4. The device in accordance with claim 1, wherein the funnel forms an angularly inclined bowl with the inlet opening being angularly inclined with respect to the open loop when oriented horizontally to hold the cup in an upright orientation.

5. The device in accordance with claim 1, wherein the inlet opening and the arm are oriented at an obtuse angle between approximately 130 to 180 degrees.

6. The device in accordance with claim 1, wherein the inlet opening and the arm are oriented at an obtuse angle between approximately 140 to 170 degrees.

7. The device in accordance with claim 1, wherein the inlet opening and the arm are oriented at an obtuse angle between approximately 150 to 160 degrees.

8. The device in accordance with claim 1, wherein the inlet opening and the open loop are oriented at an acute angle between approximately 30 to 50 degrees.

9. The device in accordance with claim 1, wherein the inlet opening and the open loop are oriented at an acute angle of approximately 40 degrees.

10. The device in accordance with claim 1, wherein the inlet opening faces upwardly and forwardly with respect to the open loop when oriented horizontally corresponding to an upright orientation of a urine cup.

11. The device in accordance with claim 1, wherein the inlet opening and the outlet opening are oriented at acute angle with respect to one another.

12. The device in accordance with claim 1, wherein the arm is oriented substantially vertically or inclined, up to 20 degrees from vertical when the open loop is oriented horizontally.

13. The device in accordance with claim 1, wherein the funnel and the arm share a wall extending between the arm and the open loop and extending through the funnel.

14. The device in accordance with claim 1, further comprising a spar extending from the funnel to the open loop with the open loop pendent from the spar, and thus the funnel.

15. The device in accordance with claim 1, further comprising an intermediate bow formed in the arm between the handle and the funnel and forming a concave arc with respect to the handle and the funnel and defining a belly indentation.

16. The device in accordance with claim 1, wherein the open loop comprises a pair of fingers; and wherein each finger forms a serpentine arc extending inwardly and outwardly along the arc from a proximal attached end to a distal free end.

17. The device in accordance with claim 1, wherein the arm extends from the handle and opens into the funnel.

18. The device in accordance with claim 1, further in combination with a sample cup carried in the open loop and with an opening of the sample cup spaced-apart from the outlet opening of the funnel.

19. A urine sample collection device, comprising:
a) an elongated arm having a proximal end defining a handle and an opposite distal end;
b) a funnel extending from the arm at the distal end and having a solid conical wall circumscribing a larger inlet opening, and having a smaller outlet opening;
c) an open loop extending from the arm at the distal end, under and spaced-apart from the outlet opening of the funnel, and having an opening therein, the open loop configured to removably receive a urine cup through the opening and hold the urine cup underneath and spaced-apart from the outlet opening of the funnel;
d) the funnel being irregular and forming an oblong frustum with a top of the frustum having the inlet opening and a bottom of the frustum being substantially closed, and with the outlet opening formed in a side of the frustum;
e) the funnel forming an angularly inclined bowl with the inlet opening being angularly inclined with respect to the open loop when oriented horizontally, the inlet opening and the open loop being oriented at an acute angle between approximately 30 to 50 degrees;
f) the inlet opening and the arm being oriented at an obtuse angle between approximately 130 to 180 degrees;
g) the arm being oriented substantially vertically or inclined, up to 20 degrees from vertical with respect to the open loop when oriented horizontally;
h) the funnel and the arm sharing a wall extending between the arm and the open loop and extending through the funnel;
i) a spar extending from the funnel to the open loop with the open loop pendent from the spar, and thus the funnel;
j) an intermediate bow formed in the arm between the handle and the funnel and forming a concave arc with respect to the handle and the funnel, and defining a belly indentation;
k) the open loop comprising a pair of fingers, each finger forming a serpentine arc extending inwardly and outwardly along the arc from a proximal attached end to a distal free end; and
l) the funnel and the open loop being sized and shaped to form a sanitary gap configured to resist contact between the sample cup and genital tissue.

20. A urine sample collection device in combination with a urine sample cup, the device comprising:
a) an arm having a handle, a funnel and an open loop;
b) the urine sample cup being removably carried by the open loop; and
c) the open loop being disposed under and spaced-apart from the funnel with a sanitary gap formed between the urine sample cup and the funnel.
